(19) United States
(12) Patent Application Publication
(10) Pub. No.: US 2026/0174664 A1
(43) Pub. Date: Jun. 25, 2026

(54) COSMETIC CLEANSING PASTE FOR SKIN BRIGHTENING

(71) Applicant: L'OREAL, Paris (FR)

(72) Inventors: Catherine CHIOU, Saddle Brook, NJ (US); Hungta LIN, Teaneck, NJ (US); Mariko HASEBE, New York, NY (US)

(73) Assignee: L'OREAL, Paris (FR)

(21) Appl. No.: 18/990,776

(22) Filed: Dec. 20, 2024

(51) Int. Cl. A61K 8/67; A61K 8/365; A61K 8/368; A61K 8/60; A61Q 19/02; A61Q 19/10

(52) U.S. Cl. CPC A61K 8/675; A61K 8/365; A61K 8/368; A61K 8/602; A61Q 19/02; A61Q 19/10

(57) ABSTRACT

A cleansing skin brightening cleansing composition includes a blend of hydroxy acids including at least one alpha-hydroxy acid, at least one beta-hydroxy acid, and at least one poly-hydroxy acid niacinamide (vitamin B3), at least one a bio-based glycolipid surfactant, at least one thickener, at least one clay, and a cosmetically acceptable solvent that includes at least water. The composition has a pH in a range from about 4.4 to about 5.0. The composition can be used as a daily skin cleanser, or a facial mask or a spot treatment product, and/or the combinations thereof, and when applied to skin confers gentle exfoliation, skin brightening and whitening, and evens skin tone.

### TECHNICAL FIELD

The present disclosure is directed to a skin care composition, in particular, a skin brightening cleansing composition having a paste-like texture.

### BACKGROUND

There is a need in the market for skin brightening cleansing compositions that have beneficial levels of actives provided in a base that allows ease of application to skin and reliable retention on targeted areas until desired removal at which time the composition may be reliably removed.

The inventors provide here a product formulation in the form of a skin brightening cleansing composition with paste-like texture that maintains stability with a high amount of skin brightening actives, is easy to spread under shear, has an aesthetically pleasing structure without dripping after application and until removal.

### SUMMARY

In various embodiments, the disclosure provides a skin brightening cleansing composition that gives rise to skin brightening effect to its users. The cleansing composition described herein is a skin brightening cleansing composition that comprises a blend of hydroxy acids including at least one alpha-hydroxy acid, at least one beta-hydroxy acid, and at least one poly-hydroxy acid, niacinamide (vitamin B3), at least one a bio-based glycolipid surfactant, at least one thickener, at least one clay, and a cosmetically acceptable solvent comprising at least water, wherein the composition has a pH in a range from about 4.4 to about 5.0, and wherein the composition has a paste-like consistency and can be used as a daily skin cleanser, a facial mask a spot treatment product, or a combinations thereof, and wherein the composition when applied to skin confers gentle exfoliation, skin brightening and whitening, and evens skin tone.

In some embodiments, the skin brightening cleansing composition comprises a blend of hydroxy acids including at least one alpha-hydroxy acid, at least one beta-hydroxy acid, and at least one poly-hydroxy acid, niacinamide (vitamin B3), at least one a bio-based glycolipid surfactant, at least one thickener, at least one clay, and a cosmetically acceptable solvent comprising at least water, wherein the pH in the range from about 4.4 to about 5.0 can minimize the generation of niacin as a degraded byproduct of niacinamide, and the composition demonstrates stability characterized by an off-white paste-like texture that does not break and maintains viscosity that is not less than about 30,000 cP, or not less than 75,000 cP, or not less than about 85,000 cP, or in a range from about 30,000 cP to greater than 300,000 cP, or in a range from about 85,000 cP to greater than 300,000 cP, or in a range from about 85,000 cP to about 300,000 cP, after storage at 45° C. fora period of two months, and wherein the composition has a paste-like consistency and can be used as a daily skin cleanser, a facial mask a spot treatment product, or a combinations thereof, and wherein the composition when applied to skin confers gentle exfoliation.

In an embodiment, the skin brightening cleansing composition comprises:
a) a hydroxy acid blend comprising at least one alpha hydroxy acid, at least one beta hydroxy acid and at least one poly hydroxy acid;
b) niacinamide;
c) at least one bio-based glycolipid surfactant;
d) at least one thickener;
e) at least one clay;
f) at least one surfactant; and
g) at least one cosmetically acceptable solvent comprising water,
wherein the skin brightening cleansing composition has a pH in a range from about 4.4 to about 5.0.

In another embodiment, the skin brightening cleansing composition comprises:
a) a hydroxy acid blend comprising at least one alpha hydroxy acid comprising lactic acid, at least one beta hydroxy acid comprising salicylic acid, and at least one poly hydroxy acid comprising gluconolactone;
b) niacinamide;
c) at least one bio-based glycolipid surfactant comprising a rhamnolipid;
d) at least one thickener comprising xanthan gum;
e) at least one clay comprising kaolin;
f) at least one surfactant comprising sodium cocoyl isethionate and glyceryl stearate SE;
g) at least one cosmetically acceptable solvent comprising water and glycerin; and
h) at least one additive selected from the group consisting of cetyl alcohol, sodium hydroxide, trisodium ethylenediamine disuccinate, magnesium aluminum silicate, pentylene glycol, butylene glycol, and combinations thereof,
wherein the skin brightening cleansing composition has a pH in a range from about 4.4 to about 5.0.

In another embodiment, the skin brightening cleansing composition comprises
a) a hydroxy acid blend comprising at least one alpha hydroxy acid comprising lactic acid, at least one beta hydroxy acid comprising salicylic acid, and at least one poly hydroxy acid comprising gluconolactone, the hydroxy acid blend present at about 4.7%;
b) niacinamide present at about 2%;
c) at least one bio-based glycolipid surfactant comprising a rhamnolipid present at about 3%;
d) at least one thickener comprising xanthan gum present at about 0.5%;
e) at least one clay comprising kaolin present at about 10%;
f) at least one surfactant comprising sodium cocoyl isethionate present at about 10% and glyceryl stearate SE present at about 5%;
g) at least one cosmetically acceptable solvent comprising water and comprising glycerin present from about 10% to about 30%; and
h) at least one additive selected from the group consisting of cetyl alcohol, sodium hydroxide, trisodium ethylenediamine disuccinate, magnesium aluminum silicate, pentylene glycol, butylene glycol, and combinations thereof,
all amounts by weight, based on the weight of the skin brightening cleansing composition, wherein the skin brightening cleansing composition has a pH in a range from about 4.4 to about 5.0.

In another embodiment, the disclosure provides a method for cleansing skin, comprising: providing a skin brightening cleansing composition, comprising:
a) a hydroxy acid blend comprising at least one alpha hydroxy acid, at least one beta hydroxy acid and at least one poly hydroxy acid;
b) niacinamide;
c) at least one bio-based glycolipid surfactant;
d) at least one thickener;
e) at least one clay;
f) at least one surfactant;
g) at least one cosmetically acceptable solvent comprising water, and comprising glycerin; and
h) at least one additive selected from the group consisting of cetyl alcohol, sodium hydroxide, trisodium ethylenediamine disuccinate, magnesium aluminum silicate, pentylene glycol, butylene glycol, and combinations thereof,
wherein the skin brightening cleansing composition has a pH in a range from 4.4 to 5.0, and
applying the composition to skin (i) in the form of a spot treatment which is dotted onto skin and after a selected period of time rinsed off, (ii) in the form of a cleanser which is applied to skin by rubbing and after a selected period of time rinsed off, (iii) in the form of a mask which is applied to skin by rubbing and retained on the skin fora selected period of time then rinsed off, and (iv) combinations thereof.

In some embodiments, the at least one alpha hydroxy acid comprises lactic acid or a derivative thereof present from about 0.1% to about 10%, by weight, based on the weight of the skin brightening cleansing composition. In some embodiments, the at least one beta hydroxy acid comprises salicylic acid or a derivative thereof present from about 0.1% to about 2%, by weight, based on the weight of the skin brightening cleansing composition. In some embodiments, the at least one poly hydroxy acid comprises gluconolactone or a derivative thereof present from about 0.1% to about 10%, by weight, based on the weight of the skin brightening cleansing composition. In some embodiments, the hydroxy acid blend is present in in a total amount from about 0.3% to about 22%, by weight, based on the weight of the skin brightening cleansing composition. In some embodiments, the hydroxy acid blend comprises lactic acid, salicylic acid, and gluconolactone, the hydroxy acid blend present in in a total amount from about 4% to about 5%, by weight, based on the weight of the skin brightening cleansing composition.

In some embodiments, niacinamide is present from about 0.1% to about 5%, by weight, based on the weight of the skin brightening cleansing composition.

In some embodiments, the at least one bio-based glycolipid surfactant is chosen from rhamnolipids, sophorolipids, glucolipids, trehalolipids, cellobiose lipids, mannosylerythritol lipid, or a mixture thereof.

In some embodiments, the at least one bio-based glycolipid surfactant is chosen from rhamnolipids present from about 1% to about 4%, by weight, based on the weight of the skin brightening cleansing composition. In some embodiments, the at least one bio-based glycolipid surfactant comprises a rhamnolipid present at about 3%, by weight, based on the weight of the skin brightening cleansing composition.

In some embodiments, the at least one thickener is chosen from xanthan gum, carrageenan, sclerotium gum, or a combination thereof, and the at least one thickener is present in a range from about 0.1% to about 5%, by weight, based on the weight of the skin brightening cleansing composition. In some embodiments, the at least one thickener is xanthan gum present from about 0.5% to about 1.5%, by weight, based on the weight of the skin brightening cleansing composition.

In some embodiments, the at least one clay comprises kaolin. In some embodiments, the at least one clay comprises kaolin present from about 0.1% to about 20%, by weight, based on the weight of the skin brightening cleansing composition.

In some embodiments, the at least one surfactant comprises sodium cocoyl isethionate, glyceryl stearate SE or a combination thereof. In some embodiments, the at least one surfactant comprises sodium cocoyl isethionate and glyceryl stearate SE present in a total amount from about from about 10% to about 20%, by weight, based on the weight of the skin brightening cleansing composition. In some embodiments, the at least one surfactant comprises sodium cocoyl isethionate, glyceryl stearate SE or a combination thereof, each present from about 1% to about 15%, by weight, based on the weight of the skin brightening cleansing composition.

In some embodiments, glycerin is present from about 10% to about 30%, by weight, based on the weight of the skin brightening cleansing composition.

In some embodiments, the skin brightening cleansing composition comprises at least one additive selected from the group consisting of anti-microbials, chelating agents, oils, fillers, penetrants, sequestrants, fragrances, dispersants, skin care actives, and combinations thereof. In some embodiments, the skin brightening cleansing composition comprises at least one additive selected from the group consisting of cetyl alcohol, sodium hydroxide, trisodium ethylenediamine disuccinate, magnesium aluminum silicate, pentylene glycol, butylene glycol, and combinations thereof. In some embodiments, the skin brightening cleansing composition comprises at least one additive comprising cetyl alcohol, sodium hydroxide, trisodium ethylenediamine disuccinate, magnesium aluminum silicate, pentylene glycol, and butylene glycol.

Other features and advantages of the present invention will be apparent from the following more detailed description of embodiments which illustrate, by way of example, the principles of the invention.

### DETAILED DESCRIPTION

The disclosure relates to compositions for acne care and methods of using the compositions.

In various embodiments, the composition is a gentle skin brightening product provided in a paste-like format that can be used as one or more of a cleanser for washing, a mask that is left in place for a selected interval, or a leave on spot treatment, each intended for short term use and removal with at least water. As used herein, “paste” and “paste-like” refers to a format that is thick/viscous and moist, having a texture that is creamy, sticky, malleable, or a combination of these, and clings without running or dripping.

According to the invention, the skin brightening cleansing composition is provided in a versatile product having a paste-like texture with a thick consistency for leave on use.

As a mask or facial cleanser to cover large areas, the skin brightening cleansing composition demonstrates minimum good spreadability and remains in place without dripping. The skin brightening cleansing composition can also be applied and retained without dripping on selected spot areas where brightening is desired.

As described in the embodiments hereof, the associations of organic acids, specifically hydroxy acids that include alpha-, beta- and poly-hydroxy acids, niacinamide and glycolipid biosurfactants (rhamnolipids) were formulated by the inventors to provide gentle skin peeling (aka exfoliation) effect, thus achieving skin brightening benefit to its users. A surprising requirement for stabilization was needed to ensure that the hydroxy acids ingredients are provided at a sufficiently low pH of the final formulation without causing skin irritation, in particular to avoid unwanted niacin production as the degraded byproduct when using niacinamide at lower pH conditions. Accordingly, the inventors unexpectedly found that the combination of xanthan gum as a thickener with a clay (such as kaolin) provide a stable and aesthetically pleasing composition in a non-dripping paste-like format which confers measurable and statistically significant skin brightening effect after use.

As shown in the examples, the inventive composition having a pH in the range from about pH 4.4 to about pH 5.0 and the combination of high amounts of hydroxy acids together with xanthan gum and clay demonstrate stability characterized by an off-white paste-like texture that does not break (i.e., no release of droplets, precipitation, cracking or thinning) and maintains viscosity that is not less than about 30,000 cP, or not less than 75,000 cP, or not less than about 85,000 cP, or in a range from about 30,000 cP to greater than 300,000 cP, or in a range from about 85,000 cP to greater than 300,000 cP, or in a range from about 85,000 cP to about 300,000 cP, after storage at 45° C. for a period of two months. Generally, the inventive composition has a viscosity in a range from about 85,000 cP to about 300,000 cP measured using a Brookfield Ameteck DV-1 Viscometer, T-F #96 Spindle, 10 rpm, 1 min. The inventive composition elicited statistically lower total fluorescence scores relative to those of untreated control sites at Day 7 and Day 23 (final evaluation) and cell turnover required fewer days on treated sites compared to untreated sites. Further the inventive composition was deemed by test users to have overall favorable attributes of texture and skin feel, with greater versatility as compared to commercial benchmarks that do not have the paste-like composition and may be used only as cleansers and not as a mask or spot treatment.

### Compositions

### Hydroxy Acids:

In various embodiments, the skin brightening cleansing composition includes at least one alpha-, at least one beta- and at least one poly-hydroxy acid. In some embodiments, the hydroxy acid blend is present in in a total amount from about 0.3% to about 22%, by weight, based on the weight of the skin brightening cleansing composition. In some embodiments, the hydroxy acid blend comprises lactic acid, salicylic acid, and gluconolactone, the hydroxy acid blend present in in a total amount from about 4% to about 5%, by weight, based on the weight of the skin brightening cleansing composition.

### Alpha Hydroxy Acid

In various embodiments, the skin brightening cleansing composition comprises at least one alpha hydroxy acid.

Suitable alpha hydroxy acids include lactic acid, glycolic acid, tartaric acid, mandelic acid, citric acid, ester derivatives thereof and combinations thereof. Exemplary ester derivatives include ester compounds of glycolic acid, lactic acid, such as methyl lactate, ethyl lactate, butyl lactate and, similarly, ester compounds of glycolic acid, tartaric acid, mandelic acid, citric acid. One particularly suitable alpha hydroxy acid is lactic acid. In some embodiments, lactic acid, or 2-hydroxypropanoic acid, is provided to the chemical peel composition to provide enhanced exfoliation of the skin. In addition, lactic acid also boosts production of glycosaminoglycan (GAG) in the skin, improving the barrier function and moisturization of skin.

In some particular embodiments, the alpha hydroxy acid in the skin brightening cleansing composition comprises lactic acid.

The skin brightening cleansing composition includes a concentration of alpha hydroxy acid in a range from about 0.1% to about 10%, or from about 1% to about 8%, or from about 1.5% to about 3%, or from about 2% to about 2.5%, or any suitable combination, sub-combination, range, or sub-range thereof by weight, based on the weight of the skin brightening cleansing composition. One of ordinary skill in the art, however, will appreciate that other ranges are within the scope of the invention. In some embodiments, the amount of alpha hydroxy acid present about 2%.

Thus, any one of or a combination of alpha hydroxy acid is present, by weight, based on the total weight of the skin brightening cleansing composition, from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, 3.0, 3.5, 4.0. 4.5, 5.0, 5.5, 6.0, 6.5, 7.0, 7.5, 8.0, 8.5, 9.0, 9.5, to about 10 weight percent, including increments and ranges therein and there between.

### Beta Hydroxy Acid

In various embodiments, the skin brightening cleansing composition comprises at least one beta hydroxy acid. In some particular embodiments, the skin brightening cleansing composition may include a beta hydroxy acid comprising salicylic acid.

The term “beta-hydroxy acid” is understood to mean, according to the present invention, a carboxylic acid having a hydroxyl functional group and a carboxylic functional group separated by two carbon atoms. A beta hydroxy acid can be present in the skin brightening cleansing composition in the form of the free acid and/or in the form of one of its associated salts (salts with an organic base or an alkali metal, in particular), especially according to the final pH imposed on the skin brightening cleansing composition.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

And in some particular embodiments, the beta hydroxy acid in the skin brightening cleansing composition excludes at least one of salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid.

In various embodiments, the skin brightening cleansing composition includes beta hydroxy acid present in a range from about 0.1% up to and not more than about 2% of beta hydroxy acid, by weight, based on the total weight of the skin brightening cleansing composition.

In some embodiments, the skin brightening cleansing composition includes from about 0.1% to about 1% of beta hydroxy acid, or from about 0.2% to about 2.0%, or from about 0.1% to about 1.5%, or from about 0.2% to about 1.5%, or from about 0.3% to about 1.0%, or from about 0.35% to about 0.75%, or from about 0.4% to about 0.5%, or is about 0.45%, or any suitable combination, sub-combination, range, or sub-range thereof by weight, based on the weight of the skin brightening cleansing composition. One of ordinary skill in the art, however, will appreciate that other ranges are within the scope of the invention.

In some embodiments, the skin brightening cleansing composition includes up to and not more than about 2%, or about 1.9% of beta hydroxy acid. In some embodiments, the skin brightening cleansing composition includes up to and not more than about 1% of beta hydroxy acid. In some embodiments, the skin brightening cleansing composition includes up to and not more than about 0.5% of beta hydroxy acid. In some embodiments, the amount of beta hydroxy acid present is not more than about 0.40% to about 0.50%.

Thus, the at least one beta hydroxy acid, when present, is present in the skin brightening cleansing composition from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

### Poly Hydroxy Acid

In various embodiments, the skin brightening cleansing composition comprises at least one poly hydroxy acid.

Suitable poly hydroxy acids include gluconolactone and derivatives thereof and combinations thereof. In some embodiments, the at least one poly hydroxy acid comprises gluconolactone or a derivative thereof present from about 0.1% to about 10%, by weight, based on the weight of the skin brightening cleansing composition.

The skin brightening cleansing composition includes poly hydroxy acid present in a range from about 0.1% to about 10%, or from about 1% to about 8%, or from about 1.5% to about 3%, or from about 2% to about 2.5%, or any suitable combination, sub-combination, range, or sub-range thereof by weight, based on the weight of the skin brightening cleansing composition. One of ordinary skill in the art, however, will appreciate that other ranges are within the scope of the invention. In some embodiments, the amount of poly hydroxy acid present about 2%.

Thus, any one of or a combination of poly hydroxy acid is present, by weight, based on the total weight of the skin brightening cleansing composition, from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, 3.0, 3.5, 4.0. 4.5, 5.0, 5.5, 6.0, 6.5, 7.0, 7.5, 8.0, 8.5, 9.0, 9.5, to about 10 weight percent, including increments and ranges therein and there between.

### Bio-Based Glycolipid Surfactant:

The composition according to the invention comprises at least one or more bio-based glycolipid surfactants. In some embodiments, the at least one bio-based glycolipid surfactant is chosen from rhamnolipids, sophorolipids, or mixtures thereof.

In some embodiments, the skin brightening cleansing composition includes only one glycolipid comprising rhamnolipid, for example sold under the name Rheance One™ by Evonik (INCI name: glycolipids), present from about 1% to about 4%, or from about 2% to about 3%, or about 3%.

The term “glycolipid” is understood as meaning a compound formed from a lipid to which are attached one or more sugar compounds.

The at least one or more glycolipids may be selected from rhamnolipids, sophorolipids, glucolipids, trehalolipids, cellobiose lipids, mannosylerythritol lipid, and mixtures thereof.

### Glucolipids:

The at least one or more glycolipids may be glucolipids, which contain a glucose moiety and can be represented by the general formula (I):

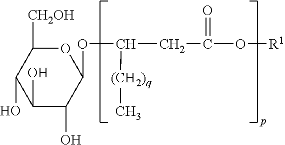

in which:
   R1 represents a hydrogen atom or a cation,
   p denotes an integer ranging from 1 to 4, and
   q denotes an integer ranging from 4 to 10, in some embodiments, equal to 6.

The glucolipids can be produced by the bacterium Alcaligenes sp. MM1.

The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids:

The at least one or more glycolipids may be sophorolipids, which contain a sophorose moiety and can be represented by the general formula (II):

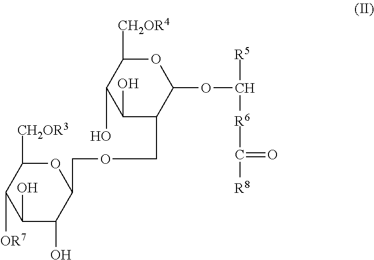

in which:
   R3 and R4 individually represent a hydrogen atom or an acetyl group,
   R5 represents a saturated or unsaturated, hydroxylated or non-hydroxylated hydrocarbon group having from 1 to 9 carbon atoms, in some embodiments, methyl,
   R6 represents a saturated or unsaturated, hydroxylated or non-hydroxylated hydrocarbon group having from 1 to 19 carbon atoms, with the proviso that the total number of carbon atoms in the groups R5 and R6 does not exceed 20 and is in some embodiments, from 14 to 18.

Sophorolipids may be incorporated into the composition according to the invention either in the form of the open-chain free acid, where R7 represents a hydrogen atom and R8 represents a hydroxy group OH, or in its lactone form, where a lactone ring is formed between R7 and R8, as indicated by formula (III):

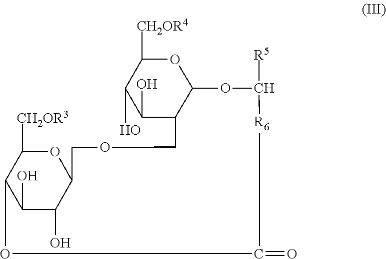

in which:
   R3, R4, R5 and R6 are as defined above,
with the proviso that at least one of R3 and R4 represents an acetyl group.

The sophorolipids can be produced by yeast cells, for example Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids for example that sold under the Sopholiance S name by Givaudan and that sold under the BioToLife name by BASF.

### Trehalolipids:

The at least one or more glycolipids may be trehalolipids, which contain a trehalose fragment and can be represented by the general formula (IV):

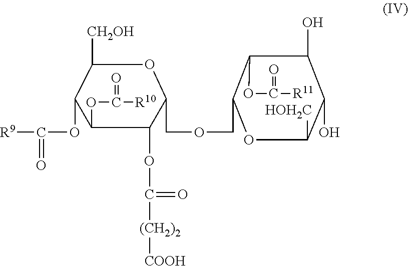

in which:
   R9, R10 and R11 individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids:

The at least one or more glycolipids may be cellobiose lipids, which contain a cellobiose fragment and can be represented by the general formula (V):

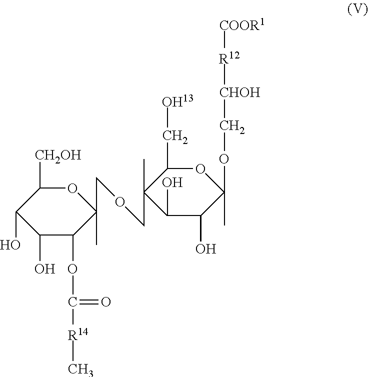

in which:
   R1 represents a hydrogen atom or a cation,
   R12 represents a saturated or unsaturated, hydroxylated or non-hydroxylated hydrocarbon radical having from 9 to 15 carbon atoms, in some embodiments, 13 carbon atoms,
   R13 represents a hydrogen atom or a acetyl group;
   R14 represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The at least one or more glycolipids may be rhamnolipids.

The composition according to the invention in some embodiments, comprises at least one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of p-hydroxylated fatty acids linked to one another by an ester bond.

More specifically, these mono-rhamnolipids and di-rhamnolipids correspond to the following formula (VI):

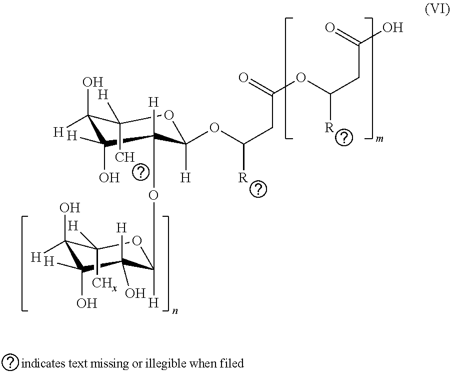

in which:
   m denotes an integer equal to 2, 1 or 0,
   n denotes an integer equal to 1 or 0, and
   R1 and R2, each independently represent identical or different hydrocarbon radicals having from 2 to 24 carbon atoms, in some embodiments, from 5 to 13 carbon atoms, that are branched or unbranched, substituted or unsubstituted, in particular hydroxy-substituted, and saturated or unsaturated, in some embodiments, a singly, doubly or triply unsaturated alkyl radical.

Thus, when n is equal to 0, the formula (VI) protects mono-rhamnolipids and, when n is equal to 1, it protects di-rhamnolipids.

The composition according to the invention in some embodiments, comprises at least one di-rhamnolipid.

The composition according to the invention in some embodiments, comprises at least one di-rhamnolipid of formula (VI) in which:
m denotes an integer equal to 2, 1 or 0;
n denotes an integer equal to 1; and
R1 and R2, each independently represent identical or different hydrocarbon radicals having from 2 to 24 carbon atoms, in some embodiments, from 5 to 13 carbon atoms, that are branched or unbranched, substituted or unsubstituted, in particular hydroxy-substituted, and saturated or unsaturated, in some embodiments, a singly, doubly or triply unsaturated alkyl radical, and also the salts thereof, solvates thereof and optical isomers thereof.

The glycosidic bond between the two rhamnose fragments may be in the alpha or beta configuration and is in some embodiments, in the alpha configuration.

In the context of the invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids in some embodiments, being in the form of the R enantiomers, and
the term “alkyl” radical denotes a saturated, linear or branched aliphatic group; for example, a C1-C20 alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention in some embodiments, comprises at least one di-rhamnolipid of formula (VI) in which:
m denotes an integer equal to 2, 1 or 0;
n denotes an integer equal to 1; and
R1 and R2, which are identical or different, are selected from pentenyl, hexenyl, heptenyl, octenyl, nonenyl, decenyl, undecenyl, dodecenyl and tridecenyl radicals and radicals of formula —(CH2)oCH3, with o denoting an integer ranging from 1 to 23, in particular from 3 to 15 and more particularly from 4 to 12.

According to one embodiment of the invention, the composition according to the invention comprises at least one di-rhamnolipids of general formula (VI) in which m is equal to 1

According to one embodiment of the invention, the composition according to the invention comprises a mixture of at least two, in some embodiments, at least three, di-rhamnolipids of general formula (VI) in which m is in some embodiments, equal to 1.

According to another embodiment of the invention, the composition according to the invention comprises a mixture comprising at least one mono-rhamnolipid.

In some embodiments, the composition according to the invention comprises at least one dirhamnolipid of the following formula (VII):

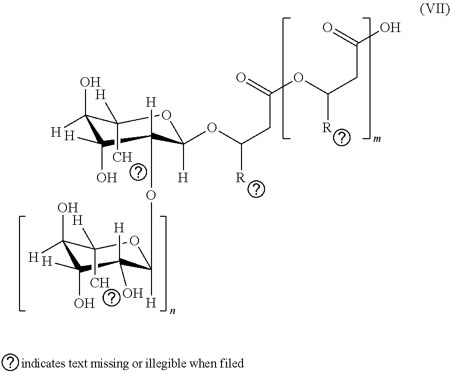

in which:
   m denotes an integer equal to 2, 1 or 0; in some embodiments, m is equal to 1,
   n denotes an integer equal to 1,
   R1 is a —(CH2)p-CH3 radical, with p being an integer varying from 1 to 23, in some embodiments, from 4 to 12,
   R2 is a —(CH2)q-CH3 radical, with q being an integer varying from 1 to 23, in some embodiments, from 4 to 12,
and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R1 and R2 respectively represented by the symbols CX and CY, the integers X and Y being respectively equal to p+4 and q+4.

TABLE 1
di-rhamnolipids of Formula (VII)

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

In some embodiments, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

In some embodiments, the di-rhamnolipid of formula (VII) in which p and q are identical and equal to 6 and m is equal to 1 is present in the composition according to the invention in a proportion of at least 50% by weight and in some embodiments, of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R1 represents a —(CH2)oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; in some embodiments, R1 represents a —(CH2)6CH3 radical and R2 a nonenyl radical.

According to another embodiment, the composition according to the invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R1 represents a —(CH2)oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; in some embodiments, R1 represents a —(CH2)6CH3 radical and R2 a nonenyl radical.

In some embodiments, the composition according to the invention comprises a mixture of at least two, in particular at least three, di-rhamnolipids of formula (VI) or of formula (VII) selected from:
at least 50% by weight and in some embodiments, of from 51% to 85% by weight of a di-rhamnolipid of formula (VII) in which p and q are identical and equal to 6 and m is equal to 1, relative to the total weight of rhamnolipids.
from 0.5% to 25% by weight, in some embodiments, from 5% to 15% by weight, of a dirhamnolipid of formula (VII) in which p is equal to 6, q is equal to 8 and m is equal to 1, relative to the total weight of rhamnolipids, and
from 0.5% to 15% by weight, in some embodiments, from 3% to 12% by weight, in some embodiments, from 5% to 10% by weight, of a dirhamnolipid of formula (VI) in which n and m are equal to 1, R1 represents a —(CH2)6CH3 radical and R2 represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

In some embodiments, the concentration of the at least one bio-based glycolipid surfactant is from about 0.1% to about 5%, or from about 1% to about 4%, or from about 2% to about 3%, or about 3%, or any suitable combination, sub-combination, range, or sub-range thereof by weight, based on the weight of the skin brightening cleansing composition. One of ordinary skill in the art, however, will appreciate that other ranges are within the scope of the invention.

Thus, the at least one bio-based glycolipid surfactant is present, by weight, based on the total weight of the skin brightening cleansing composition, from about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 to about 5 weight percent, including increments and ranges therein and there between.

### Thickener:

In various embodiments, the skin brightening cleansing composition comprises at least one thickener. The thickener may be thickening polymers. Non-limiting examples of thickening polymers include xanthan gum, sclerotium gum, carrageenan, acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, xanthan gum, modified xanthan gum, biosaccharide gum, chitin, levan, elsinan, collagen, gelatin, zein, gluten, soy protein, casein, and mixtures thereof.

In some embodiments, the skin brightening cleansing composition comprises xanthan gum present in a range from about 0.1% to about 5%, or at about 1%, by weight of the skin brightening cleansing composition.

In some embodiments, the skin brightening cleansing composition comprises xanthan gum as a particularly preferred thickener, present at about 0.5%, by weight of the skin brightening cleansing composition.

The at least one thickener, is present in the skin brightening cleansing composition from about 0.1% to about 5%, or from about 0.2% to about 3%, or from about 0.5% to about 2%, or about 1%, or any suitable combination, sub-combination, range, or sub-range thereof by weight, based on the weight of the skin brightening cleansing composition. One of ordinary skill in the art, however, will appreciate that other ranges are within the scope of the invention.

Thus, the at least one thickener may be present, by weight, based on the total weight of the skin brightening cleansing composition, from about 0.1, 0.2, 0.3, 0.4, 0.5, 0.6, 0.7, 0.8, 0.9, 1.0, 2, 3, 4 to about 5 weight percent, including increments and ranges therein and there between.

In some embodiments, the skin brightening cleansing composition excludes any one or more of alginates/algin, chitosan, chitin, starches, celluloses, galactomannans such as guar gums and its derivatives, including hydroxypropyl guar, cationic guar derivatives, gums of microbial origin, including scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, and combinations thereof.

In some embodiments, the skin brightening cleansing composition may include or exclude a nature-based polymer or synthetic polymer that is a cationic polymer.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyornithine), peptide/protein polymers ((e.g., histone, collagen), linear or branched synthetic polymers (e.g., polybrene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

In some examples, synthetic polymers are non-silicone based, and in some embodiments, may be selected from the group consisting of polyquaterniums, that is polymers having quaternary ammonium centers in the polymer, for example, including polyquaternium-47 (1-Propanaminium, N,N,N-trimethyl-3-[(2-methyl-1-oxo-2-propenyl)amino]-, chloride, polymer with methyl 2-propenoate and 2-propenoic acid).

Other non-limiting examples of polyquaternium compounds include diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53).

Other non-limiting examples of such polyquaternium compounds include Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof.

Synthetic polymer may include cationic acrylic polymers, for example, an amorphous functional acrylic polymer grafted onto a polyethylene backbone such as SYNTRAN™ 5330, which is a quaternary modified olefin grafted technology.

Synthetic polymers include, polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof.

### Clay:

In various embodiments the skin brightening cleansing composition comprises at least one clay. In some particular embodiments, the at least one clay is kaolin present from about 5% to about 20%, by weight of the skin brightening cleansing composition.

In some embodiments, if present, the at least one clay may be selected from the group of clays consisting of kaolinites, smectite, sodium smectite, calcium smectite, illite, chlorite, vermiculite, attapulgite, and combinations thereof. In some particular embodiments, the at least one clay may be kaolin. In some particular embodiments, the at least one clay may be a smectite clay in the mica family of phyllosilicates. And in some particular embodiments, the at least one clay may comprise hectorite.

In some embodiments, the at least one clay is present in an amount from about 0.1% to about 20%, or from about 5% to about 15%, or about 10%, or any suitable combination, sub-combination, range, or sub-range thereof by weight, based on the weight of the skin brightening cleansing composition.

Thus, when at least one clay is present in the skin brightening cleansing composition it may be present by weight, based on the total weight of the skin brightening cleansing composition, from about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, to about 20 percent, including increments and ranges therein and there between.

### Surfactant:

In various embodiments, the skin brightening cleansing composition comprises at least one surfactant. In some embodiments, the at least one surfactant comprises one of sodium cocoyl isethionate, glyceryl stearate SE, or a combination thereof. In some embodiments, the skin brightening cleansing composition may include, or alternatively may be substantially free from, or exclude any one or more of nonionic, cationic, or anionic surfactants. In some embodiments, the skin brightening cleansing composition excludes all surfactants except one of sodium cocoyl isethionate, glyceryl stearate SE, or a combination thereof.

The concentration of the at least one surfactant is present in the skin brightening cleansing composition from about 1% to about 20%, or from about 2% to about 18%, or from about 4% to about 15%, or from about 5% to about 10%, or about 10%, or about 5%, or any suitable combination, sub-combination, range, or sub-range thereof by weight, based on the weight of the skin brightening cleansing composition. One of ordinary skill in the art, however, will appreciate that other ranges are within the scope of the invention.

In some embodiments, the at least one surfactant includes a combination of surfactants, which together are present in a total amount in the skin brightening cleansing composition from about 1% to about 30%, or from about 10% to about 20%, or any suitable combination, sub-combination, range, or sub-range thereof by weight, based on the weight of the skin brightening cleansing composition. One of ordinary skill in the art, however, will appreciate that other ranges are within the scope of the invention.

When present, sodium cocoyl isethionate is present in the skin brightening cleansing composition is from about 1% to about 20%, or from about 2% to about 18%, or from about 5% to about 15%, or from about 8% to about 12%, about 10%, or any suitable combination, sub-combination, range, or sub-range thereof by weight, based on the weight of the skin brightening cleansing composition. One of ordinary skill in the art, however, will appreciate that other ranges are within the scope of the invention.

When present, glyceryl stearate SE is present in the skin brightening cleansing composition is from about 1% to about 20%, or from about 2% to about 15%, or from about 3% to about 10%, or from about 4% to about 6%, or about 5%, or any suitable combination, sub-combination, range, or sub-range thereof by weight, based on the weight of the skin brightening cleansing composition. One of ordinary skill in the art, however, will appreciate that other ranges are within the scope of the invention.

Thus, each of the at least one surfactant is present, by weight, based on the total weight of the skin brightening cleansing composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 18 to about 20 weight percent, including increments and ranges therein and there between.

When present, an additional surfactant, for example, but not limited to, glyceryl stearate, is present in the skin brightening cleansing composition is from about 1% to about 20%, or from about 2% to about 18%, or from about 5% to about 15%, or from about 8% to about 12%, or from about 2% to about 6%, or about 5%, or any suitable combination, sub-combination, range, or sub-range thereof by weight, based on the weight of the skin brightening cleansing composition. One of ordinary skill in the art, however, will appreciate that other ranges are within the scope of the invention.

Thus, an additional surfactant, when present, is present, by weight, based on the total weight of the skin brightening cleansing composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 18 to about 20 weight percent, including increments and ranges therein and there between.

In some embodiments, the skin brightening cleansing composition may exclude or be devoid of ethylene glycol (PEG) based surfactants or of sulfate/sulfonate based surfactant, or combinations thereof. Some specific but non-limiting examples of surfactants that may be lacking from the skin brightening cleansing composition includes those with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific surfactants that are lacking from the cosmetic skin brightening cleansing composition include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers; Acrylates/C10-30 Alkyl Acrylate Crosspolymer and similar polymeric emulsifiers.

In some embodiments, the skin brightening cleansing composition may include or exclude an amphoteric surfactant, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

### Cosmetically Acceptable Solvent:

In various embodiments, the skin brightening cleansing composition comprises at least one cosmetically acceptable solvent. In various embodiments, the cosmetically acceptable solvent may be chosen from water.

### Water:

In various embodiments, water is present in the skin brightening cleansing composition in a range from about 10% to about 60%, or from about 20% to about 55%, or from about 25% to about 50%, or about 50%, or any suitable combination, sub-combination, range, or sub-range thereof by weight, based on the weight of the skin brightening cleansing composition. One of ordinary skill in the art, however, will appreciate that other ranges are within the scope of the invention.

Thus, water may be present by weight, based on the weight of the skin brightening cleansing composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as zinc, copper, magnesium, etc., reconstituting the characteristics of a thermal water.

The skin brightening cleansing composition has a pH 4 to 4.5.

The pH may be adjusted to the desired value by addition of a base (organic or inorganic), for example sodium hydroxide, potassium hydroxide, or another suitable base, or combinations thereof.

### Water-Soluble Solvents:

In some embodiments, the skin brightening cleansing composition includes at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

In some particular embodiments, the water-soluble solvent includes glycerin present from about 5% to about 30%, or from about 10% to about 30%, by weight of the skin brightening cleansing composition.

As examples of organic solvents, non-limiting mentions can be made of ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols, glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-soluble solvents include alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In various embodiments the amount of the at least one water-soluble solvent is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the skin brightening cleansing composition. One of ordinary skill in the art, however, will appreciate that other ranges are within the scope of the invention.

In some embodiments, the skin brightening cleansing composition includes more than one water soluble solvent, each water soluble solvent present in an amount as set forth herein above, wherein each different water soluble solvent may be present within one of the ranges selected from the ranges set forth herein above.

Thus, water-soluble solvents may be present by weight, based on the total weight of the skin brightening cleansing composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Optional Additives:

In some embodiments, there may be one or more optional actives or other ingredients (herein, “additives”) present in the skin brightening cleansing composition, the one or more additives optionally selected from: anti-microbials; chelating agents; oils; fillers; penetrants; sequestrants; fragrances; dispersants; skin care actives such as ceramides, for example, sodium lauroyl lactylate (and) ceramide NP (and) ceramide AP (and) phytosphingosine (and) cholesterol (and) xanthan gum (and) carbomer (and) ceramide EOP; opacifiers; alpha-hydroxy acids; organic and inorganic UV filters; citric acid; phenylethyl resorcinol; hydroxypropyl tetrahydropyrantriol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum); retinol; niacinamide; and vitamins and vitamin derivatives, such as tocopherol and ascorbic acid; and combinations thereof.

Although the aforementioned optional additives are given as an example, it will be appreciated that other optional components compatible with cosmetic applications known in the art may be used. And of course, any one of the aforementioned additives may be excluded.

In some embodiments, the skin brightening cleansing composition includes at least one additive comprising niacinamide present from about 0.1% to about 5%, by weight, based on the weight of the skin brightening cleansing composition. In some embodiments, niacinamide is present at about 2%, by weight, based on the weight of the skin brightening cleansing composition.

In some embodiments, the skin brightening cleansing composition includes at least one additive selected from the group consisting of cetyl alcohol, sodium hydroxide, niacinamide, trisodium ethylenediamine disuccinate, and a combination thereof.

In various embodiments, the amounts of additives, for example, actives and other components, that may be present in the skin brightening cleansing composition can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the skin brightening cleansing composition.

In some embodiments, the skin brightening cleansing composition includes additives comprising cetyl alcohol (2.5%), sodium hydroxide (0.3-0.5%), niacinamide (2%), trisodium ethylenediamine disuccinate (0.5-1%), and pentylene glycol (1%) and butylene glycol (1%).

Thus, one or a combination of additives may be present in the skin brightening cleansing composition, by weight, based on the weight of the skin brightening cleansing composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### EXAMPLES

The following examples are intended to be non-limiting and explanatory in nature only. In the Examples, amounts are expressed in percentage by weight (wt %) of active materials, relative to the total weight of the composition.

### Example 1: Raw Materials

Percentages of each ingredient as may be exemplified in the following composition examples are shown as amount of raw materials, wherein the raw materials may be present in an amount that is equal to the amount of active, or if the raw material has a concentration of active that is less than 100%, then the cosmetic composition includes the raw material that includes active and a suitable solvent, wherein the concentration of active in the raw material is provided herein below in Table 2. When referring to the inventive and comparative examples, it should be presumed that each ingredient as shown in the exemplified composition is final amount of active unless otherwise indicated.

TABLE 2
Select Raw Materials (including RMs having active concentrations of less than 100%)

| | | % | |
|---|---|---|---|
| | | active | |
| Chemical Class | INCI Name | in RM | Notes |
| Alpha-Hydroxy Acid | LACTIC ACID | 90% | Active Combination |
| Beta-Hydroxy Acid | SALICYLIC ACID | 100% | |
| Poly-Hydroxy Acid | GLUCONOLACTONE | 100% | |
| Vitamin B3 | NIACINAMIDE | 100% | |
| Biosurfactant | GLYCOLIPIDS | 45% | |
| Clay | KAOLIN | 100% | Key Ingredients for |
| Thickener | MAGNESIUM ALUMINUM SILICATE | 100% | Paste Structure |
| | (Veegum HV) | | |
| Thickener | XANTHAN GUM | 100% | |
| Solid Surfactant | SODIUM COCOYL ISETHIONATE | 100% | |

### Example 2: Inventive and Comparative Compositions (all Amounts Shown in RM)

TABLE 3
Inventive Composition Examples A-C

| | | Ex. A | Ex. B | Ex. C |
|---|---|---|---|---|
| Phase | INCI Name | wt % | wt % | wt % |
| A1 | WATER/AQUA (QS) | QS to 100% | QS to 100% | QS to 100% |
| A1 | MAGNESIUM ALUMINUM | 3 | 2 | 1 |
| | SILICATE (Veegum HV) | | | |
| A1 | XANTHAN GUM | 0.5 | 0.5 | 0.5 |
| A2 | GLYCERIN | 7 | 7 | 7 |
| A2 | BUTYLENE GLYCOL | 1 | 1 | 1 |
| A2 | PENTYLENE GLYCOL | 1 | 1 | 1 |
| A2 | SALICYLIC ACID | 0.45 | 0.45 | 0.45 |
| A2 | LACTIC ACID (90% active) | 2.22 | 2.22 | 2.22 |
| A2 | GLUCONOLACTONE | 2 | 2 | 2 |
| A3 | SODIUM HYDROXIDE | QS | QS | QS |
| | (qs to pH 4.4-5.0) | | | |
| B | KAOLIN | 10 | 10 | 10 |
| C | GLYCERYL STEARATE SE | 5 | 5 | 5 |
| C | CETYL ALCOHOL | 2.5 | 2.5 | 2.5 |
| C | SODIUM COCOYL ISETHIONATE | 10 | 10 | 10 |
| C | TRISODIUM ETHYLENEDIAMINE | 1 | 1 | 1 |
| | DISUCCINATE | | | |
| D | NIACINAMIDE | 2 | 2 | 2 |
| D | GLYCOLIPIDS (45% active) | 3.33 | 3.33 | 3.33 |
| | Total (wt %): | 100 | 100 | 100 |
| | Appearance: | Off-white, thick paste | | |
| | pH (at 25° C.): | 4.41 | 4.52 | 4.64 |
| | Viscosity (Brookfield)*, cP: | 286,000 | 101,000 | 85,000 |
| | Bulk Stability (45° C., 2 months): | PASS | PASS | PASS |

*Brookfield Ameteck DV-1 Viscometer, T-F #96 Spindle, 10 rpm, 1 min

TABLE 4
Comparative Composition Examples D-J

| | | Ex. D | Ex. E | Ex. F | Ex. G | Ex. H | Ex. I | Ex. J |
|---|---|---|---|---|---|---|---|---|
| Phase | INCI Name | wt % | wt % | wt % | wt % | wt % | wt % | wt % |
| A1 | WATER/AQUA (QS) | QS to 100% | | | | | | |
| A1 | MAGNESIUM ALUMINUM | 3 | | | | | 0 | 3 |
| | SILICATE (Veegum HV) | | | | | | | |
| A1 | XANTHAN GUM | 0.5 | | | | | 0.5 | 0.5 |
| A1 | CARRAGEENAN | | 1 | 1 | 1 | 1 | | |
| A2 | GLYCERIN | 7 | 7 | 7 | 7 | 7 | 7 | 7 |
| A2 | BUTYLENE GLYCOL | 1 | 1 | 1 | 1 | 1 | 1 | 1 |
| A2 | PENTYLENE GLYCOL | 1 | 1 | 1 | 1 | 1 | 1 | 1 |
| A2 | SALICYLIC ACID | 0.45 | 0.45 | 0.45 | 0.45 | 0.45 | 0.45 | 0.45 |
| A2 | LACTIC ACID (90% active) | 2.22 | 2.22 | 2.22 | 2.22 | 2.22 | 2.22 | 2.22 |
| A2 | GLUCONOLACTONE | 2 | 2 | 2 | 2 | 2 | 2 | 2 |
| A3 | SODIUM HYDROXIDE | QS | QS | QS | QS | QS | QS | QS |
| | (qs to pH 4.4-5.0) | | | | | | | |
| B | KAOLIN | 10 | 10 | 15 | 15 | 18.75 | 10 | 0 |
| C | GLYCERYL STEARATE SE | 5 | 5 | 5 | 5 | 2.5 | 5 | 5 |
| C | CETYL ALCOHOL | 2.5 | 2.5 | 2.5 | 2.5 | 1.25 | 2.5 | 2.5 |
| C | SODIUM COCOYL | 10 | 10 | 10 | 5 | 5 | 10 | 10 |
| | ISETHIONATE | | | | | | | |
| C | TRISODIUM | 1 | 1 | 1 | 1 | 1 | 1 | 1 |
| | ETHYLENEDIAMINE | | | | | | | |
| | DISUCCINATE | | | | | | | |
| D | NIACINAMIDE | 0 | 2 | 2 | 2 | 2 | 2 | 2 |
| D | GLYCOLIPIDS (45% active) | 0 | 3.33 | 3.33 | 3.33 | 3.33 | 3.33 | 3.33 |
| | Total (wt %): | 100 | 100 | 100 | 100 | 100 | 100 | 100 |

| Appearance: | Off-white, Soft Cream | | | | | | |
|---|---|---|---|---|---|---|---|
| pH (at 25° C.): | 4.57 | 4.4 | 5.03 | 4.63 | 4.44 | 4.46 | 4.62 |
| Viscosity (Brookfield), cP: | 80,000 | 88,000 | 32,000 | 28,000 | 27,500 | 35,000 | 17,000 |
| Bulk Stability | Borderline | FAIL | FAIL | FAIL | FAIL | FAIL | FAIL |
| (45° C., 2 months): | | | | | | | |

*Brookfield Ameteck DV-1 Viscometer, T-F #96 Spindle, 10 rpm, 1 min

All composition examples were prepared following the process outlined below. Sprinkle dry blend of Veegum and xanthan gum into water while mixing with heating at 70-80° C. for 10-15 min, as needed under vacuum to prevent splashing. Add A2 ingredients, one at a time. Continue mixing while heating, until all solids dissolved and became homogeneous. Add A3 to adjust pH to 4.4-5.0. Continue mixing while heating, until all solids dissolved. Add Kaolin (Phase B) to water phase (A1A2A3) and mix well, until homogeneous. Add C ingredients, one at a time, mix until all the solids are melted and emulsified while maintaining at 75-80° C. Stop heating and start cooling. When the batch reaches 45-50° C., add D. Mix well. Continue cooling to room temperature and carefully pull vacuum. Adjust pH, if needed. Drop the batch and take specifications (pH and Viscosity).

Stability of compositions is demonstrated by an off-white paste-like texture that does not break (i.e., no release of droplets, precipitation, cracking or thinning) and maintains viscosity in a range from about 85,000 cP after storage at 45° C. for a period of two months. Generally, inventive compositions have a viscosity in a range from about 85,000 cP to about 300,000 cP measured using a Brookfield Ameteck DV-1 Viscometer, T-F #96 Spindle, 10 rpm, 1 min.

### Example 3: Cell Turnover Test

Dansyl Chloride clinical test were conducted in which Inventive Composition A and Composition D showed statistically significant improvement on “increase rate of cell turnover”, aka skin exfoliation, through the use of a fluorescent dye (Dansyl Chloride).

The objective of this cell turnover study was to determine the effect of a test material on epidermal cell exfoliation and cell turnover through the use of a fluorescent dye (dansyl chloride). Dansyl chloride has been reported to bind tenaciously to the amino groups and thus is useful for fluorescent tagging of protein. The disappearance of this fluorescent marker from the stratum corneum is an indicator of movement of cells through the epidermis and therefore a measure of the rate of cell proliferation.

Study participants were pre-treated with dansyl chloride 24 hours before applying test material. Apply a layer of the test material (Compositions A and D) sufficient to cover the test areas to the designated test sites on the volar surface of the designated right or left forearm, 1 application daily. The contralateral forearm remained untreated throughout the study, as a control site. Subjects were instructed to return to the test center for a daily evaluation for the presence of fluorescence using a Wood's lamp and continue daily test material application to the designated forearm for approximately three consecutive weeks.

A total of 33 female subjects completed the study, and the results are summarized in Table 5.

TABLE 5
Cell Turnover over Time based on Dansyl Chloride Fluorescence

| | | | | | | |
|---|---|---|---|---|---|---|
| Composition | Claim | Assessment | Day 3 | Day 5 | Day 7 | Day 23 |

| | | | | | |
|---|---|---|---|---|---|
| A | Increase | Total Cumulative | X | X | X |
| D | rate of | Fluorescence Score | | X | X |
| | cell | with Dansyl Chloride | | | |
| | turnover | labeling* | | | |

X indicates statistically significant improvement
*When compared to control (p ≤ 0.05).

Under the conditions of this study, test sites treated with Inventive Composition A and Composition D both elicited statistically lower total fluorescence scores relative to those of untreated control sites at Day 7 and Day 23 (final evaluation). Cell turnover required fewer days on treated sites compared to untreated sites. Further, Composition A showed statistically improvement starting on Day 5, better performed on cell turnover than Composition D.

### Example 4: Texture Evaluation (Consumer Study)

Texture Workshop Consumer Test was conducted and the formula was well received for its unique texture signaling effectiveness, especially appreciated as a facial mask.

Thirty-four (34) volunteers of age range of 18-45, even mix of skin type (dry, normal and oily), and range of skin tones, were recruited for a consumer study. The consumers were selected based on the following criteria: highly engaged in skincare category and are seeking skin brightening as a benefit and also interested in targeting dark spots, uneven skin tone, dull skin, and/or hyperpigmentation. The volunteers were instructed to use the test product (Inventive Composition A) at home for 1 week and to provide feedback and use experience afterwards. The objective of the study is to understand consumers' perception and experience on this multi-use product, where the test product can be used as (1) daily cleanser, (2) rinse-off face mask and (3) overnight spot treatment. The overall liking by test panelists is summarized as below in Table 6.

TABLE 6
Consumer Preference

| Strongly Disliked | Disliked | Neutral | Liked | Loved |
|---|---|---|---|---|
| 0 | 3 | 4 | 17 | 9 |

In addition, panelists have ranked favorably on the following attributes of Composition Example A (using score of 1-5, 5 being the most favorable): texture (4.6), feel on skin as a cleanser (4.0), feel on skin as a mask (4.6), feel on skin as a spot treatment (3.8), scent (3.7) and overall rating (4.5).

In summary, consumers have appreciated this unique, multi-use formula and found it more versatile to use than their usual products.

### Example 5: Brightening Effects Evaluation (Consumer Study)

Consumer Home-Use Test (4-week usage). Inventive Composition A provides subtle brightening benefits within the first week and improvement over the 4-week testing period, and is regarded as generally safe for all skin types.

A four (4)-week study was carried out with a monadic design, participants were selected based on the same criteria stated in Example 4 of Texture Evaluation, where Group 1 (29 participants) used Composition Example A; and Group 2 (29 participants) were given 2 commercial products to be used as a combined cleansing and treatment regimen (both products were blinded with generic labels). Both groups were instructed to use the test products at home replacing their current cleaning and treatment products. Since there were no multi-use products as disclosed in this invention in the market, two separate products were used as a routine and served as the performance benchmark. The commercial products used in this study were Murad Environmental Shield™ Essential-C™ Cleanser and Murad Environmental Shield™ Dark Spot Correcting Serum.

At the end of 4-week usage at home, Group 2 (the benchmark group) has reported that while thoroughly enjoyed the routine, the products do not offer the unique convenience of multi-use. While participants from Group 1 reported enjoyable application of using Composition Example A and appreciated the routine simplicity and versatility of this multi-use product.

While the disclosure has been described with reference to specific embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

### Definitions and Terms

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material.

The term “consisting of” excludes any element, step, or material other than those specified in the claim and, in the latter instance, impurities ordinary associated with the specified material(s).

The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated. All percentages, parts and ratios herein are based upon the total weight of embodiments of the composition of the present disclosure, unless otherwise indicated.

“Skin care actives” are ingredients included in the formulation to provide specific benefits to the skin, such as exfoliation, brightening, and improvement of texture and tone.

“Fatty compounds” are lipophilic substances that provide moisturizing properties and enhance the emollience of the creamy brightening cleanser, contributing to skin softness and hydration.

“Additives” include a range of optional components such as preservatives, fragrances, and UV filters, which enhance the overall sensory experience and stability of the product.

“Surfactants” are surface-active agents that lower the surface tension between two substances, such as oil and water, aiding in the formation of stable emulsions and effective cleansing. As used herein, the term “surfactants,” as well as any specifically identified surfactants, includes salts of the surfactants even if not explicitly stated.

“Thickener” refers to ingredients that increase the viscosity of the creamy brightening cleanser, providing a desirable texture and stability without affecting the active components' efficacy.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the embodiments of the composition according to the disclosure or the material may be absent. For instance, there may be less than 2% by weight of a specific material added to a composition, based on the total weight of the compositions (provided that an amount of less than 2% by weight does not materially affect the basic and novel characteristics of embodiments of the composition according to the disclosure. Similarly, the compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “devoid,” “essentially free” (or “substantially free”) of one or more components that are positively set forth in the instant disclosure and a component or ingredient may be excluded from the composition. The term “substantially free” or “essentially free” as used herein may also mean that the specific material is not added to the composition but may still be present in a raw material that is included in the composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A skin brightening cleansing composition comprising: a blend of hydroxy acids including at least one alpha-hydroxy acid, at least one beta-hydroxy acid, and at least one poly-hydroxy acid, niacinamide (vitamin B3), at least one a bio-based glycolipid surfactant, at least one thickener, at least one clay, and a cosmetically acceptable solvent comprising at least water, wherein the composition has a pH in a range from about 4.4 to about 5.0, and wherein the composition has a paste-like consistency and can be used as a daily skin cleanser, a facial mask a spot treatment product, or a combinations thereof, and wherein the composition when applied to skin confers gentle exfoliation, skin brightening and whitening, and evens skin tone.

2. The skin brightening cleansing composition according to claim 1, wherein the at least one alpha hydroxy acid comprises lactic acid or a derivative thereof present from about 0.1% to about 10%, by weight, based on the weight of the skin brightening cleansing composition, and wherein the at least one beta hydroxy acid comprises salicylic acid or a derivative thereof present from about 0.1% to about 2%, by weight, based on the weight of the skin brightening cleansing composition, and wherein the at least one poly hydroxy acid comprises gluconolactone or a derivative thereof present from about 0.1% to about 10%, by weight, based on the weight of the skin brightening cleansing composition.

3. The skin brightening cleansing composition according to claim 1, wherein the pH in the range from about 4.4 to about 5.0 can minimize the generation of niacin as a degraded byproduct of niacinamide

4. The skin brightening cleansing composition according to claim 1, wherein the composition demonstrates stability characterized by an off-white paste-like texture that does not break and maintains viscosity in a range from about 85,000 cP to about 300,000 cP after storage at 45° C. for a period of two months.

5. The skin brightening cleansing composition according to claim 1, wherein the hydroxy acid blend is present in in a total amount from about 0.3% to about 22%, by weight, based on the weight of the skin brightening cleansing composition.

6. The skin brightening cleansing composition according to claim 1, wherein hydroxy acid blend comprises lactic acid, salicylic acid, and gluconolactone, the hydroxy acid blend present in in a total amount from about 4% to about 5%, by weight, based on the weight of the skin brightening cleansing composition.

7. The skin brightening cleansing composition according to claim 1, wherein the at least one bio-based glycolipid is chosen from rhamnolipids, sophorolipids, glucolipids, trehalolipids, cellobiose lipids, mannosylerythritol lipid, or a mixture thereof.

8. The skin brightening cleansing composition according to claim 1, wherein the at least one bio-based glycolipid comprises a rhamnolipid present from about 1% to about 4%, by weight, based on the weight of the skin brightening cleansing composition.

9. The skin brightening cleansing composition according to claim 1, wherein the at least one thickener is present in a range from about 0.1% to about 5%, by weight, based on the weight of the skin brightening cleansing composition.

10. The skin brightening cleansing composition according to claim 1, wherein the at least one thickener is xanthan gum present from about 0.5% to about 1.5%, by weight, based on the weight of the skin brightening cleansing composition.

11. The skin brightening cleansing composition according to claim 1, wherein the at least one clay comprises kaolin present from about 0.1% to about 20%, by weight, based on the weight of the skin brightening cleansing composition.

12. The skin brightening cleansing composition according to claim 1, wherein the at least one surfactant comprises sodium cocoyl isethionate, glyceryl stearate SE or a combination thereof, each present from about 1% to about 15%, by weight, based on the weight of the skin brightening cleansing composition.

13. The skin brightening cleansing composition according to claim 1, wherein the at least one surfactant comprises sodium cocoyl isethionate and glyceryl stearate SE present in a total amount from about from about 10% to about 20%, by weight, based on the weight of the skin brightening cleansing composition.

14. The skin brightening cleansing composition according to claim 1, wherein glycerin is present from about 10% to about 30%, by weight, based on the weight of the skin brightening cleansing composition.

15. The skin brightening cleansing composition according to claim 1, wherein the skin brightening cleansing composition comprises at least one additive selected from the group consisting of anti-microbials, chelating agents, oils, fillers, penetrants, sequestrants, fragrances, dispersants, skin care actives, and combinations thereof.

16. The skin brightening cleansing composition according to claim 1, wherein the skin brightening cleansing composition comprises at least one additive selected from the group consisting of cetyl alcohol, sodium hydroxide, trisodium ethylenediamine disuccinate, magnesium aluminum silicate, pentylene glycol, butylene glycol, and combinations thereof.

17. The skin brightening cleansing composition according to claim 1 wherein:
a) the hydroxy acid blend comprises at least one alpha hydroxy acid comprising lactic acid, at least one beta hydroxy acid comprising salicylic acid, and at least one poly hydroxy acid comprising gluconolactone;
b) the at least one bio-based glycolipid comprises a rhamnolipid;
c) the at least one thickener comprises xanthan gum;
d) the at least one clay comprises kaolin;
e) the at least one surfactant comprises sodium cocoyl isethionate and glyceryl stearate SE; and
f) the at least one cosmetically acceptable solvent comprises water and glycerin; and
wherein the composition comprises cetyl alcohol, sodium hydroxide, trisodium ethylenediamine disuccinate, magnesium aluminum silicate, pentylene glycol, butylene glycol, and combinations thereof.

18. A skin brightening cleansing composition comprising:
a) a hydroxy acid blend comprising at least one alpha hydroxy acid comprising lactic acid, at least one beta hydroxy acid comprising salicylic acid, and at least one poly hydroxy acid comprising gluconolactone, the hydroxy acid blend present at about 4.7%;
b) niacinamide present at about 2%;
c) at least one bio-based glycolipid comprising a rhamnolipid present at about 3%;
d) at least one thickener comprising xanthan gum present at about 0.5%;
e) at least one clay comprising kaolin present at about 10%;
f) at least one surfactant comprising sodium cocoyl isethionate present at about 10% and glyceryl stearate SE present at about 5%;
g) at least one cosmetically acceptable solvent comprising water and comprising glycerin present from about 10% to about 30%; and
h) at least one additive selected from the group consisting of cetyl alcohol, sodium hydroxide, trisodium ethylenediamine disuccinate, magnesium aluminum silicate, pentylene glycol, butylene glycol, and combinations thereof,
all amounts by weight, based on the weight of the skin brightening cleansing composition,
wherein the skin brightening cleansing composition has a pH in a range from 4.4 to 5.0.

19. A method for cleansing skin comprising:
a) providing a skin brightening cleansing composition according to claim 1;
b) applying the composition to skin (i) in the form of a spot treatment which is dotted onto skin and after a selected period of time rinsed off, (ii) in the form of a cleanser which is applied to skin by rubbing and after a selected period of time rinsed off, (iii) in the form of a mask which is applied to skin by rubbing and retained on the skin for a selected period of time then rinsed off, and (iv) combinations thereof.

20. The method according to claim 19, wherein in the provided skin brightening cleansing composition
a) the hydroxy acid blend comprises at least one alpha hydroxy acid comprising lactic acid, at least one beta hydroxy acid comprising salicylic acid, and at least one poly hydroxy acid comprising gluconolactone;
b) the at least one bio-based glycolipid comprises a rhamnolipid;
c) the at least one thickener comprises xanthan gum;
d) the at least one clay comprises kaolin;
e) the at least one surfactant comprises sodium cocoyl isethionate and glyceryl stearate SE; and
f) the at least one cosmetically acceptable solvent comprises water and glycerin; and
wherein the composition comprises cetyl alcohol, sodium hydroxide, trisodium ethylenediamine disuccinate, magnesium aluminum silicate, pentylene glycol, butylene glycol, and combinations thereof.
